(19) United States
(12) Patent Application Publication
(10) Pub. No.: US 2006/0241758 A1
(43) Pub. Date: Oct. 26, 2006

(54) Facet spacers

(75) Inventors: Marc M. Peterman, Memphis, TN (US); Jeff R. Justis, Collierville, TN (US); Fred J. Molz IV, Collierville, TN (US); Courtney S. Williams, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/110,470

(22) Filed: Apr. 20, 2005

(51) Int. Cl. A61F 2/44

(52) U.S. Cl. 623/17.11; 623/14.12

(57) ABSTRACT

A method of decompressing a facet joint comprises forming an opening in a facet capsule of the facet joint and inserting a spacer device through the opening into the facet capsule.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Disc deterioration and other spinal deterioration may cause spinal stenosis, a narrowing of the spinal canal and/or the intervertebral foramen, that causes pinching of the spinal cord and associated nerves. Current methods of treating spinal stenosis include laminectomy or facet resection. Alternative and potentially less invasive options are needed to provide spinal pain relief.

### SUMMARY

In one embodiment, a method of decompressing a facet joint comprises forming an opening in a facet capsule of the facet joint and inserting a spacer device through the opening into the facet capsule.

In another embodiment, a system for decompressing a joint comprises means for insertion into a facet capsule between adjacent articular processes. The means increases a distance between the adjacent articular processes.

In another embodiment, a device for separating a pair of articular processes comprises an implantable member adapted for insertion into a facet capsule between first and second articular processes.

In another embodiment, a system for decompressing an intervertebral joint comprises a first spacer device adapted for insertion into a first facet capsule between a first pair of articular processes.

In another embodiment, a method of decompressing a facet joint comprises locating a facet joint comprising a pair of articular processes and inserting a spacer device between the articular processes, wherein the articular processes are unresected.

### BRIEF DESCRIPTION OF THE DRAWINGS

FIG. 1 is a perspective view of a vertebral column

FIG. 2 is a sectional view of a facet joint of the vertebral column of FIG. 1.

FIG. 3 is a sectional environmental view of a facet spacer according to one embodiment of the present disclosure.

FIG. 4 is a top view of the facet spacer of FIG. 3.

FIG. 5 is a sectional view of a facet spacer within a facet joint according to another embodiment of the present disclosure.

FIG. 6. is a top view of the facet spacer of FIG. 5.

FIG. 7 is a sectional view of a facet spacer within a facet joint according to another embodiment of the present disclosure.

FIG. 8 is a sectional view of a facet spacer within a facet joint according to another embodiment of the present disclosure.

FIG. 9 is a sectional view of a facet spacer within a facet joint according to another embodiment of the present disclosure.

### DETAILED DESCRIPTION

The present disclosure relates generally to the field of orthopedic surgery, and more particularly to systems and methods for decompressing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

Referring first to FIG. 1, the numeral 10 refers to a vertebral joint which includes an intervertebral disc 12 extending between vertebrae 14, 16. The vertebra 14 includes a lamina 18, and the vertebra 16 includes a lamina 20. The vertebrae 14, 16 also include vertebral bodies 14a, 16a, respectively. The vertebra 14 further includes caudal articular processes 22, 24. The vertebra 16 further includes rostral articular process 26, 28. A facet joint 30 is formed, in part, by the adjacent articular processes 22, 26. A facet joint 32 is formed, in part, by the adjacent articular processes 24, 28. Although the illustration of FIG. 1 generally depicts the vertebral joint 10 as a lumbar vertebral joint, it is understood that the devices, systems, and methods of this disclosure may also be applied to all regions of the vertebral column, including the cervical and thoracic regions. Furthermore, the devices, systems, and methods of this disclosure may be used in non-spinal orthopedic applications.

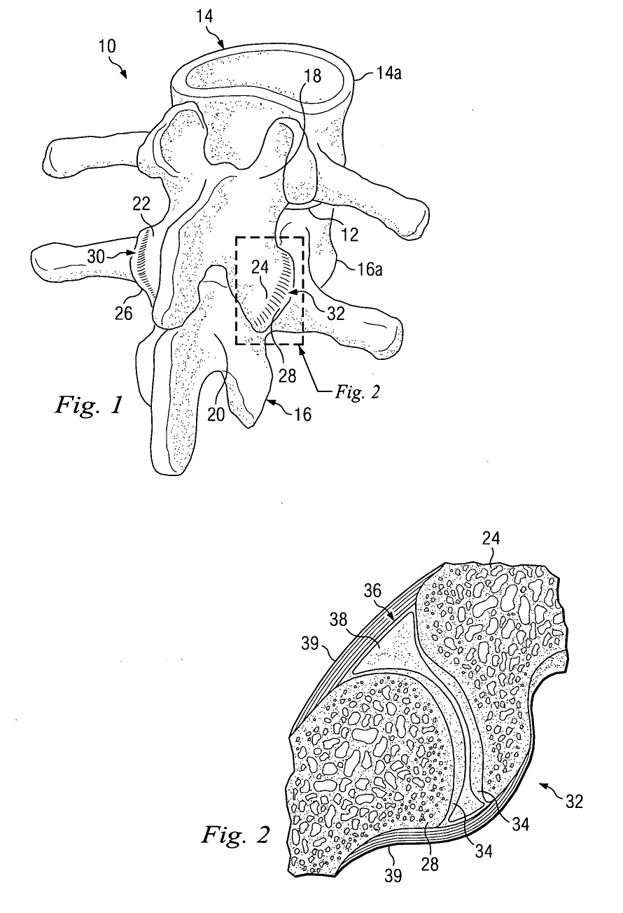

Referring now to FIG. 2, in a healthy patient, the facet joint 32, also termed zygapophyseal joint, further includes articular cartilage 34 covering articulating surfaces of the articular processes 24, 28. A facet capsule 36 within the facet joint 32 contains synovial fluid 38 which lubricates the joint 32 to decrease friction. Ligaments 39 hold the facet joint 32 together and form a portion of the facet capsule 36. The type of motion permitted by the facet joints is dependent on the region of the vertebral column. For example, in a healthy lumbar region, the facet joints limit rotational motion but permit greater freedom for flexion, extension, and lateral bending motions. By contrast, in a healthy cervical region of the vertebral column, the facet joints permit rotational motion as well as flexion, extension, and lateral bending motions. As the facet joint deteriorates, the facet capsule may become compressed and worn, losing its ability to provide a smooth, lubricated interface between the articular surfaces of the articular processes. This may cause pain and limit motion at the affected joint. Facet joint deterioration may also cause inflammation and enlargement of the facet joint which may, in turn, contribute to spinal stenosis. Traditional methods of treatment, including removal of all or a portion of an afflicted articular process, may result in abnormal motions and loading on the remaining components of the joint. The embodiments described below may be used to decompress a deteriorated facet joint and restore more natural motion.

Injury, disease, and deterioration of the intervertebral disc may also cause pain and limit motion. In a healthy intervertebral joint, the intervertebral disc permits rotation, lateral bending, flexion, and extension motions. As the intervertebral joint deteriorates, the intervertebral disc may become compressed, displaced, or herniated, resulting in excess pressure in other areas of the spine, particularly the posterior bony elements of the afflicted vertebrae such as the facet joints. The loss of disc height associated with degenerative disc disease or other disc pathologies may cause retrolisthesis in which the rostral vertebra displaces posteriorly on the caudal vertebra. This displacement may lead to spinal stenosis and may cause the superior articular process to slip posteriorly over the inferior articular process. The embodiments described below may reverse degenerative displacement of the vertebral bodies and articular processes, decompress the intervertebral disc and/or facet joint, and relieve spinal stenosis.

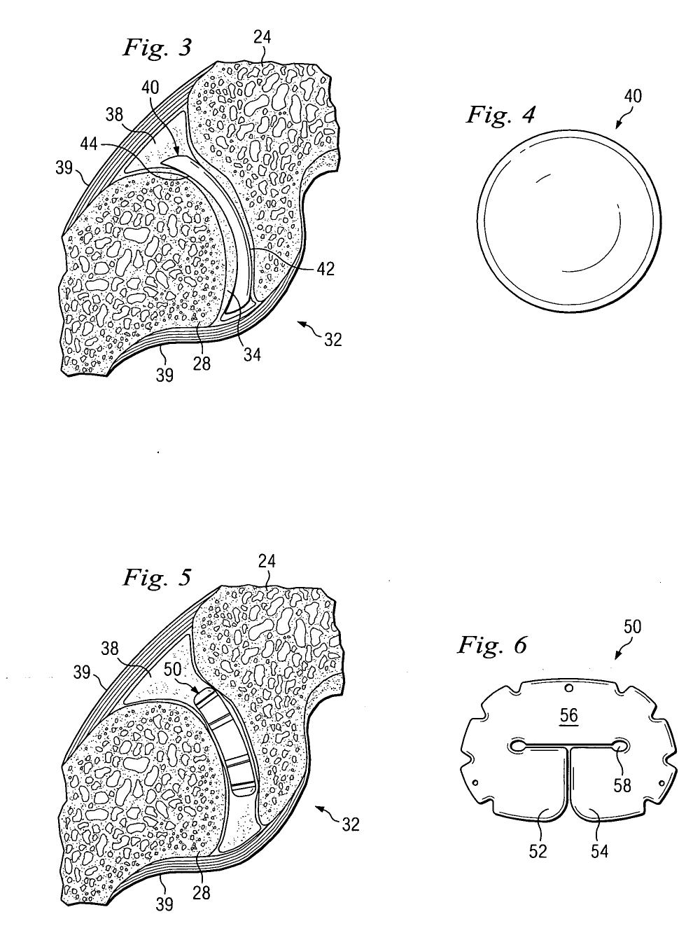

Referring now to FIGS. 3 and 4, in this embodiment, a spacer device 40 may be lens shaped, having convex faces 42, 44 which may generally match the shape of the articulating surfaces of articular processes 24, 28. The spacer device 40 be formed of a relatively rigid material to resist impact forces and to create a wedge distraction between the articular surfaces of the articular processes 24, 28. Suitable rigid materials may include metals such as cobalt-chromium alloys, titanium alloys, nickel titanium alloys, and/or stainless steel alloys. Cobalt chrome alloy, for example, may bear well on native cartilage 34 Ceramic materials such as aluminum oxide or alumnia, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

Other suitable materials for spacer device 40 may be softer, deformable, and more elastic to provide greater shock absorption. Such materials may include a wide variety of biocompatible polymeric materials including silicone, hydrogels, and polyurethane.

The spacer device 40 may be implanted between the articular processes 24, 28 and into the facet capsule 36 using a variety of techniques. In one embodiment, a surgical procedure to implant the spacer system 40 may be ultra minimally invasive. Using a posterior, posterolateral, lateral, lateral or other suitable approach, a small incision may be created in the patient's skin. A minimally invasive dilation instrument may be inserted through the incision to dilate skin and muscular tissue. During this procedure, the articular processes 24, 28 and the facet capsule 36 may be visualized directly or with radiographic assistance. A portion of the facet capsule wall and the surrounding ligaments 39 may also be dilated to create a passage into the facet capsule 36. The spacer device 40 may be passed through the dilated passage and into the facet capsule 36 such that the convex surface 42 is located adjacent to the articular surface of the articular process 24 and the convex surface 44 is located adjacent to the articular surface of the articular process 28. Although, the spacer device 40 may be in direct contact with the articular processes 24, 28, it is understood that cartilage, fluid. For example, the synovial fluid 38 may flow about the spacer device 40. With the spacer device 40 implanted, the dilated opening in the facet capsule may be allowed to contract and reseal. In some embodiments, a closure mechanism such as a suture or stopper may be used to seal the facet capsule. Although in this embodiment the facet capsule 36 has been maintained, in alternative embodiments, the facet capsule may be destroyed and the spacer device held in place by the compression of the articular processes 24, 28 and/or surrounding soft tissue.

In certain anatomies, the spacer device 40 may be used alone to provide decompression to a single targeted facet joint or to relieve pressure on a particular side of the intervertebral disc, such as a herniation area. Additionally, a second spacer device may be installed on the opposite lateral side from the spacer system 40, in the facet joint 30. The dual spacer devices may provide more balanced support and equalized decompression.

The spacer device 40, as installed, may axially separate the vertebrae 14, 16, relieving pressure on the intervertebral disc 12 and the facet joint 32 and reducing wear and further degeneration. The spacer device 40 may be load bearing and dampen the forces on the intervertebral disc 12 and articular processes 24, 28 during motion such as flexion and extension. Additionally, the spacer device 40 may decompress the neural foramen, restoring a more natural foraminal height. Any retrolisthesis of the vertebrae 14, 16 and the articular processes 24, 28 that may be developed may be corrected by the spacer device, and further migration may be prevented.

Referring now to FIGS. 5 and 6, in this embodiment, a spacer device 50 may be similar to the implants described in U.S. Pat. No. 6,620,196, entitled “Intervertebral Disc Nucleus Implants and Methods,” which is incorporated by reference herein. The spacer device 50 may fully or partially fill the facet capsule 36. The device 50 may be a load bearing elastic body having a shape memory and may be configured to allow extensive short-term manual or other deformation without permanent deformation, cracks, tears, breakage or other damage are provided. When the device 50 is in an undeformed, relaxed state, end portions 52, 54 are positioned adjacent to a central portion 56 to form at least one inner fold 58.

The spacer device 50 may be fabricated in a wide variety of load-bearing shapes capable of withstanding spinal loads and other spinal stresses. For example, the spacer device 50 may generally conform to the shape of the natural facet capsule 36 and the articular surfaces of the articular processes 24, 28. The device 50 may restore a natural spacing between the articular processes 24, 28. Alternatively, the spacer device may provide more or less spacing than natural to achieve a desired facet and/or spinal disc decompression. Although spacer device 50 is shown as having unitary construction, it may, alternatively, be made from several discrete or connected pieces.

Spacer device 50 may be formed from a wide variety of biocompatible polymeric materials, 0including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally-occurring materials such as glucomannan gel, hyaluronic acid, polysaccharides, such as cross-linked carboxyl-containing polysaccharides, and combinations thereof. The nature of the materials employed to form the elastic body of spacer device 50 may be selected so the formed implants have sufficient load bearing capacity. It is under stood that a combination of materials may be used included combinations of rigid and flexible materials.

The spacer device 50 may be implanted between the articular processes 24, 28 and into the facet capsule 36 using a variety of techniques. In one embodiment, a surgical procedure to implant the spacer system 50 may be ultra minimally invasive. Using a posterior, posterolateral, lateral, antero-lateral or other suitable approach, a small incision may be created in the patient's skin. A minimally invasive dilation instrument may be inserted through the incision to dilate skin and muscular tissue. During this procedure, the articular processes 24, 28 and the facet capsule 36 may be visualized directly or with radiographic assistance. Some distraction of the articular processes 24, 28 may be useful to access the facet capsule 36. A portion of the facet capsule wall and the surrounding ligaments 39 may be dilated to create a passage into the facet capsule 36. The spacer device 50 may be deformed or unfolded by, for example, manual force into a substantially straightened configuration for insertion through the dilated passage in the facet capsule 36. As the device 50 enters the facet capsule 36, and is no longer subject to manual force, it is allowed to deform back into its relaxed, folded configuration. Thus, the shape-memory material and folded configuration of the spacer device 50 may permit insertion through an opening smaller than the space ultimately occupied. Depending upon the size selected, the spacer device 50 can substantially fill and conform to the space between the articular processes 24, 28. Although the spacer device 50 may be in direct contact with the articular processes 24, 28, it is understood that cartilage, fluid, or other tissue may extend between the spacer device and the articular processes. For example, the synovial fluid 38 may flow about the spacer device 50. With the spacer device 50 implanted, the dilated opening in the facet capsule may be allowed to contract and reseal. In some embodiments, a closure mechanism such as a suture or stopper may be used to seal the facet capsule. Through this method of implantation, resection of the articular processes 24, 28 may be avoided or minimized, and the facet capsule 36 may be enhanced but otherwise remain generally intact and functional.

Spacer device 50 is configured to is resist expulsion or other migration through an opening or weak portion of the facet capsule and to resist excessive migration within an facet capsule. In an alternative embodiment, the surface of the spacer device may include various surface features, including various macro-surface patterns, and chemical or physical modifications to further enhance fixation of the implant within the facet capsule. In other embodiments, a flexible spacer device may be encapsulated or have a resorbable outer shell to provide additional support and/or enhance fixation. Although in this embodiment the facet capsule 36 has been maintained, in alternative embodiments, the facet capsule may be destroyed and the spacer device held in place by the compression of the articular processes 24, 28 and/or surrounding soft tissue.

In certain anatomies, the spacer device 50 may be used alone to provide decompression to a single targeted facet joint or to relieve pressure on a particular side of the intervertebral disc, such as a herniation area. Additionally, a second spacer device may be installed on the opposite lateral side from the spacer system 50, in the facet joint 30. The dual spacer devices may provide more balanced support and equalized decompression.

The spacer device 50, as installed, may axially separate the vertebrae 14, 16, relieving pressure on the intervertebral disc 12 and the facet joint 32 and reducing wear and further degeneration. The spacer device 50 may be load bearing and dampen the forces on the intervertebral disc 12 and articular processes 24, 28 during motion such as flexion and extension. Additionally, the spacer device 50 may decompress the neural foramen, restoring a more natural foraminal height. Any retrolisthesis of the vertebrae 14, 16 and the articular processes 24, 28 that may be developed may be corrected by the spacer device, and further migration may be prevented.

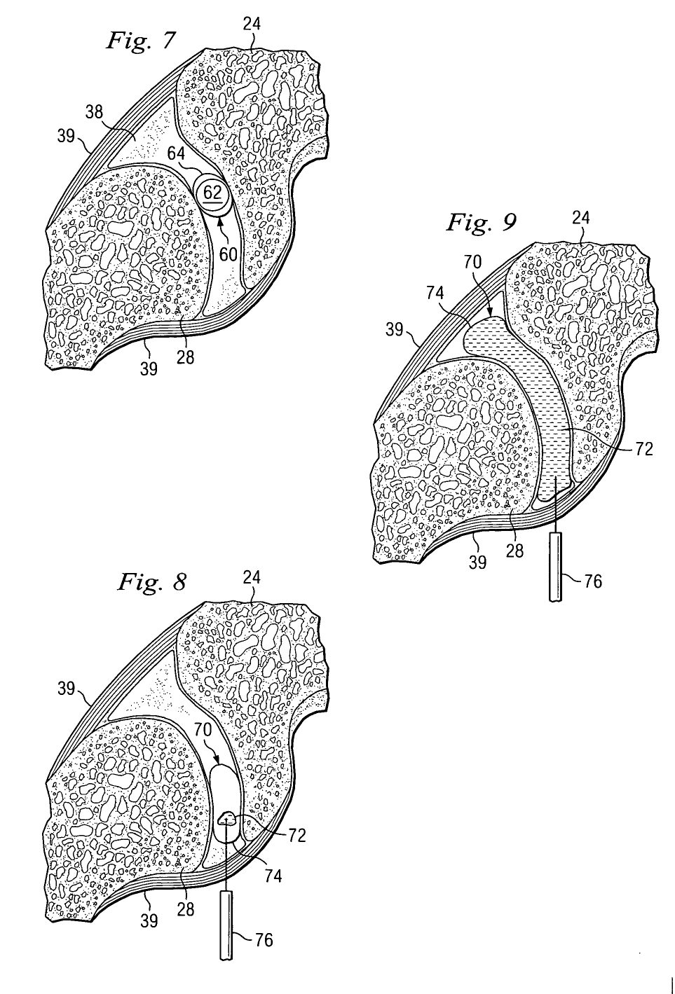

Referring now to FIG. 7, in this embodiment, a spacer system 60 may include a spherical shaped spacer device 62 and a deformable pocket 64. The spacer device 62 may be formed of a relatively rigid material to resist impact forces and to create a wedge distraction between the articular surfaces of the articular processes 24, 28. The spherical shape of the spacer device 62 may also promote articulation between the articular processes 24, 28. Suitable rigid materials may include metals such as cobalt-chromium alloys, titanium alloys, nickel titanium alloys, and/or stainless steel alloys. Cobalt chrome alloy, for example, may bear well on native cartilage 34. Ceramic materials such as aluminum oxide or alumnia, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. The pocket 64 may be formed from a biocompatible, flexible material which may be resorbable or nonresorbable.

The spacer system 60 may be installed using a method similar to that described above for device 40. In one embodiment, the pocket 64 may be inserted first to receive the spacer device 62. In an alternative embodiment, the device 62 may be inserted first and the pocket 64 inserted later to envelop the device. It is understood that in still another alternative, the insert 62 can be used without pocket. Although in this embodiment the facet capsule 36 has been maintained, in alternative embodiments, the facet capsule may be destroyed and the spacer device held in place by the compression of the articular processes 24, 28 and/or surrounding soft tissue.

The spacer device 62 of this embodiment may function similar to a miniaturized Fernstrom ball, a type of implant known to preserve height and articulation in intervertebral disc applications. In certain anatomies, the spacer system 60 may be used alone to provide decompression to a single targeted facet joint or to relieve pressure on a particular side of the intervertebral disc, such as a herniation area. Additionally, a second spacer device may be installed on the opposite lateral side from the spacer system 60, in the facet joint 30. The dual spacer devices may provide more balanced support and equalized decompression.

The spacer system 60, as installed, may axially separate the vertebrae 14, 16, relieving pressure on the intervertebral disc 12 and the facet joint 32 and reducing wear and further degeneration. The spacer system 60 may be load bearing and dampen the forces on the intervertebral disc 12 and articular processes 24, 28 during motion such as flexion and extension. Additionally, the spacer system 60 may decompress the neural foramen, restoring a more natural foraminal height. Any retrolisthesis of the vertebrae 14, 16 and the articular processes 24, 28 that may be developed may be corrected by the spacer device, and further migration may be prevented.

Referring now to FIGS. 8 and 9, in this embodiment, a spacer system 70 may include a spacer device 72 and a deformable pocket 74. The spacer device 72 may be a compliant mass of material that is curable or otherwise hardenable to form a biconvex shape similar to spacer device 40 which may generally match the shape of the articulating surfaces of articular processes 24, 28. The spacer device 72 may be formed of a relatively rigid material to resist impact forces and to create a wedge distraction between the articular surfaces of the articular processes 24, 28. Alternatively, the spacer device 72, in a cured state, may remain compliant or elastic and allow extensive short-term manual or other deformation without permanent deformation, cracks, tears, breakage or other damage.

The spacer device 72 may be a flowable substance prepared from any suitable materials. The material for spacer device 72 may include polymeric materials having a desired combination of such properties as biocompatibility, physical strength and durability, and compatibility with other components (and/or biomaterials) used in the assembly of a final composite. Examples of suitable materials for use in preparing the biomaterial include polyurethanes, hydrogels, epoxies, polysiloxanes, polyacrylates, and combinations thereof. Hardenable ceramic or other non-polymeric materials may also be suitable.

The deformable pocket 74 may be formed of an expandable or unfurlable material. The pocket may act as a balloon, dam, or retainer. Suitable materials may include elastomeric polymers such as polyurethanes. Mesh materials formed of plolyamides, polyesters, polyethylenes, polyproylenes, or other polymers may also be used. Likewise, auto- or allo-raft tissue may also be used to form the pocket.

Using a method similar to that described above for system 50, the system 70 may be implanted between the articular processes 24, 28. Specifically, a surgical procedure to implant the system 70 may be ultra minimally invasive. Using a posterior, posterolateral, lateral, antero-lateral or other suitable approach, a small incision may be created in the patient's skin. A minimally invasive dilation instrument may be inserted through the incision to dilate skin and muscular tissue. During this procedure, the articular processes 24, 28 and the facet capsule 36 may be visualized directly or with radiographic assistance. Some distraction of the articular processes 24, 28 may be useful to access the facet capsule 3 and to locate the articular processes in a desired position. A portion of the facet capsule wall and the surrounding ligaments 39 may be dilated to create a passage into the facet capsule 36. Through this passage, the compacted pocket 74 may be inserted into the facet capsule 36. An injection instrument 76 may be filled with the all or components of the material for forming spacer device 72, and inserted into the facet capsule 36. The material may be injected into the pocket 74 which may expand or unfurl to fill all or a portion of the capsule 36. The material may be allowed to cure or other wise harden to create the spacer device 72. While the material is hardening, the articular processes 24, 28 may remain held in a distracted position. Although device 72 may be in direct contact with the articular processes 24, 28, it is understood that cartilage, fluid, or other tissue may extend between the spacer device and the articular processes. For example, the synovial fluid 38 may flow about the spacer device 72 and the pocket 74. In some embodiments, a closure mechanism such as a suture or stopper may be used to seal the facet capsule. Through this method of implantation, resection of the articular processes 24, 28 may be avoided or minimized, and the facet capsule 36 may be enhanced but otherwise remain generally intact and functional.

In an alternative embodiment, the pocket may be omitted and the hardenable material injected directly into the facet capsule to cure. Although the cured spacer device 72 may be biconcave as described above, in an alternative embodiment, other geometries may be used to match contours of the articular processes 24, 28 or to create other desired distractions. In still another alternative, the unitary construction may be replaced with a series of discrete members injected into the facet capsule and allowed to fill voids, provide resurfacing, or form shapes unique to a particular patient anatomy.

The terms “rostral” and “caudal” are used in some embodiments to describe the position of components of the embodiments. While rostral is typically used in the art to describe positions toward the head and caudal is used to describe positions toward the tail or foot, as used herein, rostral and caudal are used simply as modifiers for the relative locations of components of the illustrated embodiments. For example, rostral components may be on one side of an illustrated joint, and caudal may be on another side of the joint. Components labeled as rostral or caudal to describe an illustrated embodiment are not intended to limit the orientation of a device or application of a method relative to a patient's anatomy, or to limit the scope of claims to any device or method.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “rostral,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of decompressing a facet joint, the method comprising:
forming an opening in a facet capsule of the facet joint and
inserting a spacer device through the opening into the facet capsule.

2. The method of claim 1 further comprising:
inserting a minimally invasive dilation instrument into the facet joint.

3. The method of claim 2 wherein the step of forming comprises inserting the minimally invasive dilation instrument into the facet capsule.

4. The method of claim 1 wherein the step of inserting a spacer device comprises inserting a lens shaped spacer device into the facet capsule.

5. The method of claim 1 wherein the step of inserting a spacer device comprises inserting a spherical shaped spacer device into the facet capsule.

6. The method of claim 1 wherein the step of inserting a spacer device comprises inserting an elastically deformable spacer device into the facet capsule.

7. The method of claim 1 wherein the step of inserting a spacer device comprises inserting a rigid spacer device into the facet capsule.

8. The method of claim 1 wherein the step of inserting a spacer device comprises injecting a material into the facet capsule.

9. The method of claim 8 further comprising:
distracting the facet joint as the material is injected and
distracting the facet joint as the material becomes at least partially cured.

10. The method of claim 1 further comprising:
deforming the spacer device while inserting the spacer device through the opening into the facet capsule.

11. The method of claim 1 further comprising:
providing a closure device to the opening.

12. The method of claim 1 wherein the step of inserting further comprises inserting the spacer device into synovial fluid.

13. A system for decompressing a joint, the system comprising:
means for insertion into a facet capsule between adjacent articular processes, wherein said means increases a distance between the adjacent articular processes.

14. A device for separating a pair of articular processes, the device comprising:
an implantable member adapted for insertion into a facet capsule between first and second articular processes.

15. The device of claim 14 wherein the implantable member comprises a biconvex disc.

16. The device of claim 14 wherein the implantable member is elastically deformable.

17. The device of claim 14 wherein the implantable member is adapted to take a first shape during insertion between the first and second articular processes and take a second shape after insertion between the first and second articular processes.

18. The device of claim 14 wherein the implantable member has a generally spherical shape.

19. The device of claim 18 further comprising a pocket for receiving the load-bearing member.

20. The device of claim 14 wherein the implantable member comprises an injectable and curable material.

21. The device of claim 20 further comprising a deformable pocket adapted for insertion into the facet capsule and further adapted for receiving the injectable and curable material.

22. The device of claim 14 wherein the implantable member is load bearing.

23. A system for decompressing an intervertebral joint, the system comprising:
a first spacer device adapted for insertion into a first facet capsule between a first pair of articular processes.

24. The system of claim 23 further comprising:
a second spacer device adapted for insertion into a second facet capsule between a second pair of articular processes.

25. A method of decompressing a facet joint, the method comprising:
locating a facet joint comprising a pair of articular processes; and
inserting a spacer device between the articular processes, wherein the articular processes are unresected.
